(19) United States
(12) Patent Application Publication
(10) Pub. No.: US 2003/0093089 A1
(43) Pub. Date: May 15, 2003

(54) Apparatus for variable micro abrasion of human tissue and/or hides using different size and types of abrasive particles

(76) Inventor: Ronald Allan Greenberg, Woodforde (AU)

Correspondence Address: PAUL D. GREELEY, ESQ., OHLANDT, GREELEY, RUGGIERO & PERLE, L.L.P., ONE LANDMARK SQUARE, 10th FLOOR, STAMFORD, CT, 06901-2682, US

(21) Appl. No.: 10/323,544

(22) Filed: Dec. 19, 2002

Related U.S. Application Data
(62) Division of application No. 10/110,650, filed on Aug. 12, 2002.
Application No. 10/110,650 is a 371 of international application No. PCT/AU00/01290, filed on Oct. 20, 2000.

(30) Foreign Application Priority Data
Oct. 20, 1999 (AU) PQ 3578
Jun. 1, 2000 (AU) PQ 7874

(51) Int. Cl. A61B 17/50

(52) U.S. Cl. 606/131

(57) ABSTRACT

A microabrasion apparatus for providing treatment of skin or other surfaces whereby a pneumatic source [(12, 38)] is operatively connected to a recovery container [(14, 22)] and to a hand tool [(16, 12)] and to one of a plurality of supply containers [(18, 26)] that house treatment particulates. A valve [(30)] controls which supply container [(18, 26)] provides the treatment particles. In operation, the user can select from different supply containers and thus different particles each of which performs different types of treatment such as abrasion. In addition, there is provided the use of a microabrasion apparatus using organic particulates that provide natural treatment of skin.

This application is a divisional application of U.S. application Ser. No. 10/110,650 filed on Apr. 16, 2002.

The present invention relates to an apparatus and method for variable micro-abrasion using abrasive particles of different sizes and/or type.

### BACKGROUND OF THE INVENTION

Apparatus for making micro-abrasions, particularly for cosmetic, medical or therapeutic treatment of human tissue such as the removal of scars and other skin blemishes are known. In addition, these apparatus may be used to treat hide or other similar type of materials, which require fine abrasion to remove particular attributes. Typically these apparatus are used to remove scars and other blemishes by removing surface skin layers by the use of abrasive particles or particles.

The particles are generally driven by a pneumatic source, such as a vacuum source. Such an apparatus includes a vacuum pump connected in series by tubes to a recovery container, a handle and a supply container housing abrasive particles. The handle includes an aperture which when positioned on a surface to be treated prevents any external air for entering the tubes and causes the vacuum pump to draw particles from the supply container through the handle and into the recovery container. Whilst passing through the handle the particles (or particles) impinge on the surface causing an abrasive action. The particles are then sucked into the recovery container where filters ensure that the particles remain in the recovery container.

The flow of particles depends on the flow of air through the apparatus. Initially air is drawn into the apparatus through the aperture. When the aperture is positioned against a surface to be treated then the handle becomes effectively sealed and air is evacuated from the apparatus leading to a reduction in air pressure. As more and more air is evacuated, the particle flow slows down and may eventually stop requiring the handle to be detached from the surface to allow atmospheric air to flow back into the apparatus. Not only does this lead to loss of treatment time but also the apparatus can, even when the aperture is just placed on the surface, frequently become blocked. Various systems to overcome these problems have been proposed, such as a burst of pressurised air to clear the blockages. These systems are not only more complex but cause the particles to spray outwards contaminating the surrounding area. Not only is this a nuisance, but also the fine sand-like particles may constitute a safety hazard.

To overcome this problem, patent application PCT/AU98/00936, by the present applicant proposed an apparatus that minimised blockage of the apparatus by using the exhaust port of the vacuum pump to provide a pressurised airflow into the supply container in addition to the suction form the vacuum pump. In that same application, the applicant also proposed an improved hand tool and supply container which substantially overcame these problems.

However, in some application it may be desirable to provide an apparatus that not only provides for the controlled fine micro-abrasion but one where the abrasion needs to be very intense or strong. This may be required, for example, in medical applications where it may be desirable to remove almost all of the skin layers down to the muscle and fat tissue.

To achieve dermabrasion the particles must posses a certain level of abrasiveness. Commonly used in the industry are aluminium oxide particles whose microscopic shape includes sharp points and edges that provide good abrasion against the skin.

However, for some time, there has been growing concern over the treatment of human bodies with substances that are thought to be associated with various degenerative diseases. Included in this group is the use of aluminium oxide as a dermabrasion particulate. Recently aluminium has been identified as a possible cause of Alzheimer's disease and a number of people have been concerned with its use, however slight.

The use of such abrasive particles can also leave the skin dry and does not provide the exposed skin with any nourishment or a protective layer.

It may therefore be desirable to provide for micro-abrasion using particles that not only abrade the skin but also treat it at the same time by imparting essential oils, fragrances and other substances.

It is therefore an object of the present invention to provide a micro-abrasion apparatus that provides a hitherto unknown range of micro-abrasion intensity and treatment. This is achieved by providing a micro-abrasion apparatus that may use a plurality of containers, each container having different size and/or types of particles, or by an apparatus with at least one container, where said particles used are ones that when treating the skin impart essential oils, fragrances or other substances to the skin.

### SUMMARY OF THE INVENTION

Therefore in one form of the invention there is proposed an apparatus for providing treatment of a surface such as skin including:

a pneumatic source operatively connected to a recovery container and to a hand tool;

a plurality of supply containers including particulates;

a valve selectively connecting said pneumatic source, recovery container and hand tool to a selected one of said supply containers;

whereby in operation said pneumatic source provides for an air flow through the recovery container, hand tool and the selected supply container in communication with same, drawing said. particles from the supply container through the hand tool and into the recovery container, said hand tool positioned on a surface to be treated and including an aperture so located that particles passing through said hand tool are caused to impinge on the surface thereby treating it.

I preference the apparatus includes a plurality of recovery containers whereby each supply container has a corresponding unique recovery container that it is in communication with.

Preferably the apparatus includes a plurality of hand tools each hand tool being associated with a unique supply container.

Preferably each supply container contains different particles.

Thus one can see that the apparatus provides for different supply container providing different particles and where there may be one or more recovery containers that recover those particles for subsequent disposal.

Preferably at least one supply container includes aluminium oxide particles and at least one other supply container includes aluminium oxide particles whose size is on the average greater than those in the other.

Preferably at least one said supply container includes organic type particles.

Preferably said organic type particles are made from ground up nut shells chosen from the selection including but not limited to walnut, hazelnut, brazil and almond nut shells or a mixture thereof.

Preferably said organic type particles are made from ground up stone fruit kernels chosen from the selection but not limited to peach, apricot, nectarine, peacherine, plum, cherry stones or a mixture thereof.

In preference the pneumatic source is a vacuum pump.

In preference the vacuum pump includes a regulator so as to control the air pressure within the apparatus.

Preferably said apparatus further includes a second pneumatic source selectively and operatively connected to another supply container.

Preferably said second pneumatic source is a compressor.

Preferably said compressor can variably control the air pressure fed into the supply container.

In preference there are at least three supply containers each including different particles to assist in treatment of the skin.

In preference at least some of said particles are coated with fragrant materials.

In preference at least some of said particles are coated with a colour.

In a further form of the invention there is proposed an apparatus for providing treatment of a surface including:

a pneumatic source operatively connected to a recovery container, to a hand tool, and to a supply container said supply container housing organic particulates;

whereby in operation said pneumatic source provides for an air flow through the recovery container, hand tool and the supply container in communication with same, drawing said organic particles from the supply container through the hand tool and into the recovery container, the hand tool being positioned on a surface to be treated and including an aperture so located that particles passing through said hand tool are caused to impinge on the surface thereby treating it.

Preferably said organic particles include a surface film pat of which remains in the surface being treated.

In a further aspect of the invention there is proposed a method of treatment of human skin including directing an airflow including organic particles onto the skin to be treated.

In a still further aspect of the invention there is proposed a method of treating human skin including:

using an air flow to carry particles through a hand tool, said hand tool including an aperture which is so located that upon placement on the skin the particles are caused to impinge on it and wherein said particles are organic type particles.

### BRIEF DESCRIPTION OF THE DRAWINGS

The accompanying drawings, which are incorporated in and constitute a part of this specification, illustrate several implementations of the invention and, together with the description, serve to explain the advantages and principles of the invention. In the drawings,

FIG. 1 is a perspective view of a microabrasion apparatus with two different particles that can be used including two supply and recovery assemblies and two pneumatic sources;

FIG. 2 is a schematic representation of the microabrasion apparatus of FIG. 1;

FIG. 3 is a schematic representation of a microabrasion apparatus having tow different supply and recovery assemblies but using only one pneumatic source;

FIG. 4 is a schematic representation of a microabrasion apparatus having two different particles but only one recovery container;

FIG. 5 is a schematic representation of a three supply containers having different particles with only one recovery container, and

FIG. 6 is a schematic representation of a microabrasion apparatus using only one particle type such as organic particles with only one pneumatic source.

### DESCRIPTION OF THE PREFERRED EMBODIMENTS

The following detailed description of the invention refers to the accompanying drawings. Although the description includes exemplary embodiments, other embodiments are possible, and changes may be made to the embodiments described without departing from the spirit and scope of the invention. Wherever possible, the same reference numbers will be used throughout the drawings and the following description to refer to the same and like parts.

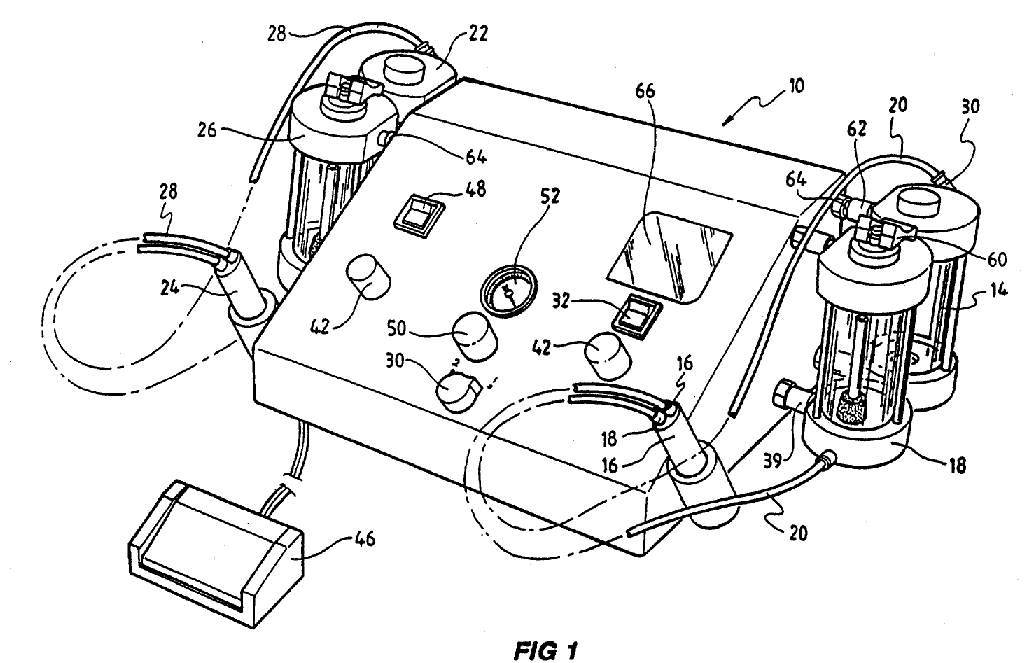

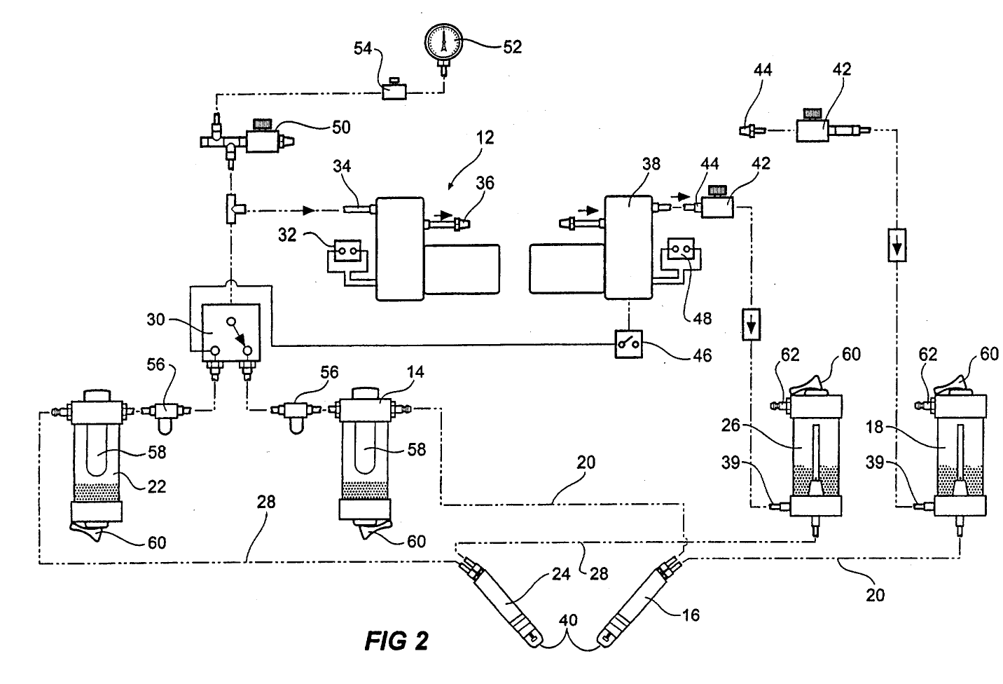

Turning now to the figures in detail, and specifically FIGS. 1 and 2, there is shown an apparatus 10 for making abrasions including a vacuum pump 12 operatively connected in series to either a first assembly including a recovery container 14, a handle 16 and a supply container 18 by tubes 20, or operatively connected to a second assembly including a recovery container 22, a handle 24 and a supply container 26 by tubes 28.

Switch 30 controls a solenoid that switches the vacuum pump from operatively being connected either to the first or second assembly. The vacuum pump is operable by an electric switch and draws air through inlet 34 and exhaust's it through outlet 36.

Second assembly supply container 26 is provided with particles that are of a different type, for example bigger (heavier), than those found in supply container 18. If the particles are heavier then they of a size so that the same density of particles provides a greater abrasive result. Because the particles are substantially heavier and to provide for a more instant particle flow it is preferably for the second assembly to include a second pneumatic source. Accordingly a compressor 38 is operatively connected to the supply container 26 or to the container that may house the heavier particles.

In operation, the vacuum pump draws air through either one of the assemblies and the corresponding recovery container and handle. The handle includes an aperture 40, which is positioned against the surface to be treated, and which then causes air and thus particles to be drawn from the corresponding recovery container. The stream of particles then impinges on the surface against which the aperture is positioned causing micro-abrasions and is subsequently drawn into the recovery container together with any abraded surface debris.

When a low-level of micro-abrasion is required the switch 30 is operated so that the solenoid operatively connects the vacuum pump in series with the first assembly that in this example houses lighter abrasive particles. In such a configuration the vacuum pump provides suction through the recovery container 14, handle 16 and supply container 18. The supply container 18 includes an inlet 39 connected to a regulator 42 having an inlet 44 the regulator controlling the amount of air entering the supply container. The air entering the supply container aerates the particles, with the magnitude of air flow providing control over the flux or density of particles drawn though the apparatus. For further details of this operation the reader is referred to the applicants patent specification referred to above.

In use where a different level or type of microabrasion is required, switch 30 operates the solenoid to operatively connect the vacuum pump 12 in series with the second assembly. The supply container 26 of the second assembly as with the first assembly also includes an inlet 40 connected to regulator 42 with an inlet 44. However, unlike the regulator used in the first assembly, the inlet 44 is operatively connected to the compressor 38. Advantageously, when the switch 30 operatively connects the vacuum pump with the second assembly, control switch 46, usually a foot control switch, controls the compressor 38 to provide pressure to the supply container 26. The foot switch may be a variable switch that variably controls the speed and thus the pressure provided by the compressor.

An isolation switch 48 can isolate the operation of (or power to) the compressor from switch 46. Similarly isolation switch 32 controls the first pneumatic source or the vacuum pump.

Thus, if one wants to use the second assembly to provide for micro abrasions using heavier particles stored in the recovery container 26 using handle 24 without the extra air flow provided by the compressor, one can turn off switch 48. But, to assist in the flow of different or heavier particles and provide a greater degree of micro-abrasion, is it advantageous to use the compressor to provide the greater flow rate and particle density.

The vacuum pump includes a suction regulator 50 that controls the suction provided to the apparatus by the vacuum pump 12. A vacuum gauge 52 advantageously provides an indicator of the vacuum within the apparatus, the vacuum gauge being isolated by the use of valve 54.

Filters 56 ensure that the particle flowing from the supply container, through the handle and into the recovery container to not enter the vacuum pump and cause potential damage. The recovery containers may also include filter 58 that prevent any particles form passing into the vacuum pump.

In use, after the vacuum pump 12 has been activated, the operator chooses whether they want to use the first assembly or second assembly by operating switch 30. The appropriate hand tool is then used with its aperture 40 positioned against a surface to be treated. Air is then drawn or sucked through the appropriate recovery container, handle and the supply container, the amount of suction regulated by regulator 50.

Regulator 42 that assists in providing aeration of the particles and as discussed in the applicant's. earlier application can control the particle flux or density.

When the second assembly is selected by use of switch 30, the foot pump is activated that allows the operator to provide pressurised air to the supply container 26, which can also be controlled by regulator 42. It is however to be understood that the regulators are not essential to the working of the invention and the compressor may simply be turned off and on by the use of the foot switch. However, to provide for fine adjustment of the particle flux or density it is advantageous to have the regulators present.

To allow access to the abrasive particles, the supply and recovery container may include quick-release lids 60 allowing access into the containers. To also assist in mounting the container to the apparatus, the recovery and the supply container may include projections 62 adapted to engage holding bores 64 on the apparatus.

Timer 66 can assist the operator in keeping a track of time that the machine has been used for. Although not shown, the times could be electrically connected to the pneumatic sources so that the apparatus turns off after a pre-determined amount of time.

Thus one can see that the present dual bottle apparatus provides for a hitherto unknown range of micro-abrasion properties by providing for different size particles that provide different degrees of micro-abrasions.

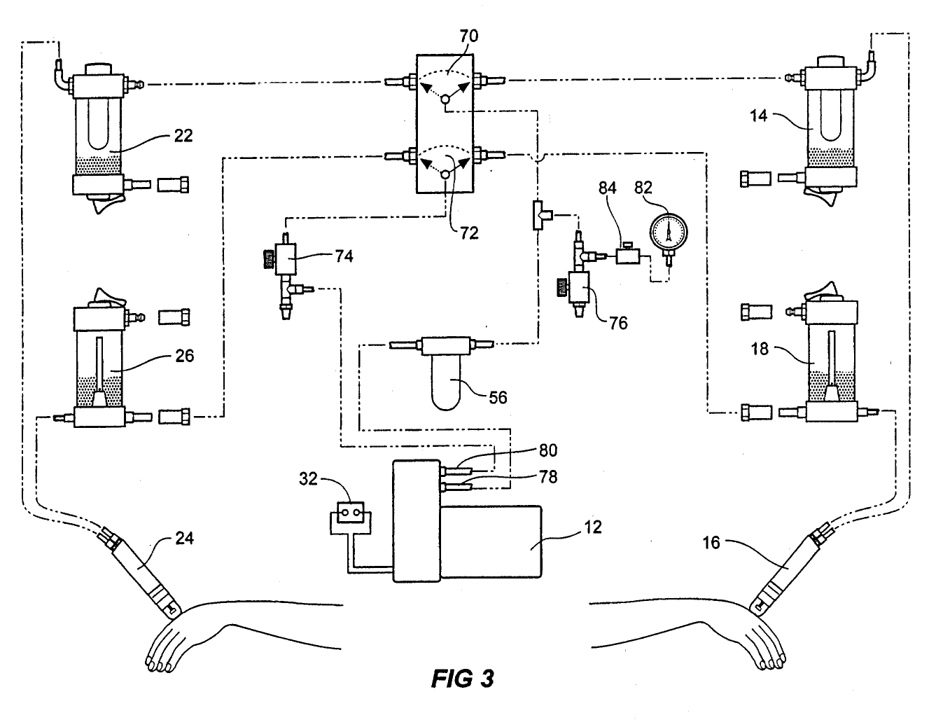

Referring now to FIG. 3 there is shown an apparatus that incudes two assemblies, that is two supply and recovery container, but only one pneumatic source. In this embodiment, there are two valves 70 and 72 that are adapted to operate in tandem and that control the coupling of the pneumatic source with the relevant supply and recovery containers.

Thus the vacuum pump 12 is either connected so as to provide a pneumatic source to recovery container 14, handle 16 and supply container 18 or alternatively to supply recovery container 22, handle 24 and supply container 26. The vacuum source in the embodiment also includes two regulators 74 and 76 that control exposure to the inlet 78 and outlet 80 of the vacuum pump. A meter 82 may also be present to provide and indication to the operator of the air pressure within the system. The meter can be isolated by valve 82. Filter 56 filters out any particulate matter form entering the vacuum pump.

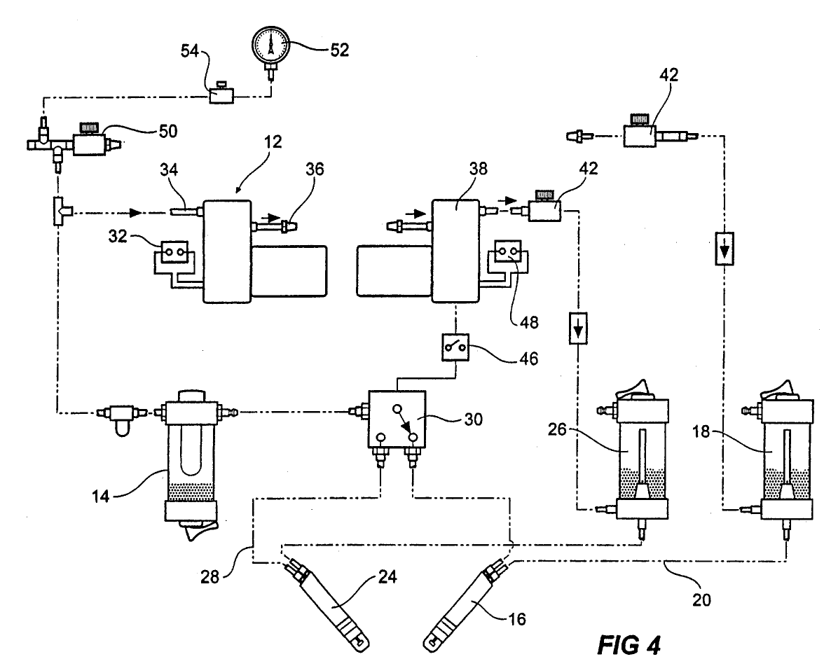

Whilst at times it may be desirable and even necessary to have independent assemblies, one may also have a joint assembly whereby a plurality of supply containers are in communication with only one recovery container. This is shown in FIG. 4 where the supply containers 18 or 26 can be coupled to the one recovery container 14 by the use of valve 30. As was the case in the previous embodiment, one selection of the switch may also turn on a second pneumatic source, such as compressor 38.

It has also been discovered that the use of hard organic material that is sufficiently dried, may be used in micro-dermabrasion apparatus as discussed above providing an effective abrasive action against the skin whilst at the same time leaving the skin relatively smooth and supple as compared with the use of aluminium oxide particles.

One of the organic materials that have been found to be particularly useful is ground up nutshells such as walnut shells. These ground up or pulverized shells can sufficiently dry not to cause blocking in the tubes of a micro-dermabrasion apparatus and yet still have levels of oils and other substances that are left as a residue against the skin on which they are impacted.

In fact, experimentation with different types of organic material has shown, that provided that the particles are sufficiently dry and of a suitable size, a whole range of organic particles may be used. For example, one may equally well use coconut shell, walnut shell, peach kernels and other fruit kernels but to name a few.

Thus a person who wishes to be treated by a certain organic material due to certain properties of that material may choose to have that material prepared in granular form and dried to be used in a micro-dermabrasion apparatus. In this way the skin may e slightly abraded at the same time providing treatment by that organic material to the satisfaction of the consumer.

It is also desirable at times to still use highly abrasive particles such as aluminium oxide. However, it has been discovered that by mixing aluminium oxide particles with organic material that may not be abrasive, one achieves not only good abrasion but also simultaneous treatment of the skin.

Accordingly one can have a choice of different particles to use in a micro-dermabrasion machine including:

(a) Purely organic materials.

(b) A mixture of abrasive particles such as aluminium oxide mixed with organic materials.

(c) Abrasive particles that are coated with organic materials.

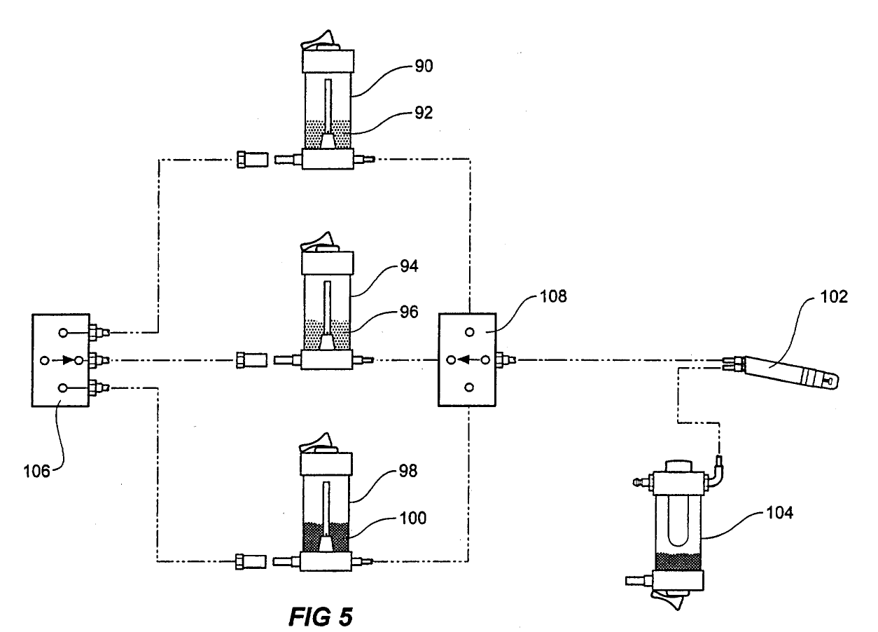

This can be achieved in the present apparatus by having an apparatus with a plurality of supply containers housing different particles. As shown in FIG. 5 for example, there may be three different supply containers. Container 90 may house standard aluminium oxide particles 92, supply container 94 may house organically coated aluminium oxide particles 96 whist supply container 98 may house organic particles 100. A supply container can be chosen to be in communication with handle 102 and recovery container 104 by the use of valve 106 that controls air flow into the respective supply container and valve 108 that than connects that supply container to the handle 102 and recovery container 104.

In some cases the micro-dermabrasion apparatus may need to be modified to accommodate organic particles. This may include the dimensions of the tubes, airflow as well as the size of the handle and aperture. Further, the supply and collection containers may also need to be modified.

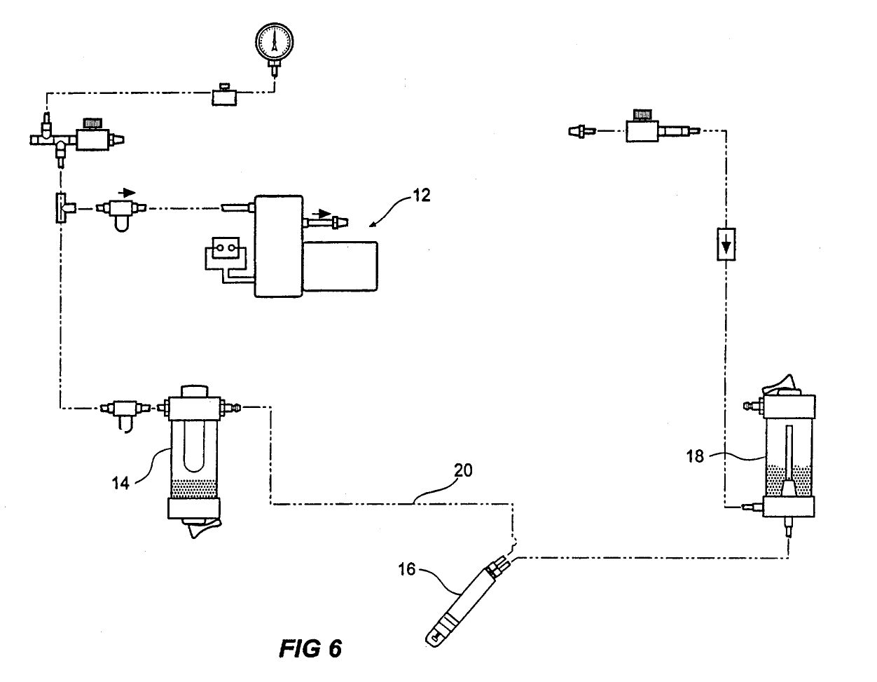

However, it may also be possible to use one apparatus where the supply container is simply changed thereby changing the abrasive particles. Thus, an apparatus such as that shown in FIG. 6 may only have one supply container 18 and recovery container 14 and only one pneumatic source 12. To use different particles, the supply container can simply be replaced at the operator's discretion.

Further, instead of having uniform size particles in the one supply container, in some cases one may wish to use a mixture of different sizes. As discussed above, one may choose to have aluminium oxide particles mixed with organic material whose size is smaller than that of the aluminium oxide.

If properly prepared the use of organic particles has been found to provide good abrasive rates. Of course, in some instance, such as medical applications it may be preferable to use aluminium oxide particles, or ones coated with an organic material.

The organic particles may not only contain essential oils but they may also contain fragrances that on impact with the skin impart some of those properties. One therefore proposes the use of particles in micro-dermabrasion machines that have been exposed to fragrances. For example, one may very well coat aluminium oxide particles with fragrant materials that impact their fragrance to the skin besides just abrading it.

It is to be understood that one does not need to just coat the abrasive particles, one may provide a mixture of abrasive particles and fragrant particles where the end result is that the abrasive particles abrade the skin whilst the fragrant articles impart the fragrances to the skin.

Of course, it may be that the apparatus contains separate abrasive and fragrant particles and that a user is firstly treated with the highly abrasive particles and subsequently with the organic ones that assist in leaving a residual layer on the skin.

A micro-dermabrasion operator may provide the consumer with a choice of mixing various abrasive particles or particles with various oils/fragrance particles which are then used in the skin treatment, Of course, great care needs to be taken to ensure that the particle do not become too wet whereupon they will not be able to move freely through the apparatus but will stick to the container and the various tubes.

The impacted skin therefore not only receives the benefits of micro-dermabrasion but also interaction with the abrasive particles, which leaves the skin with residual properties of the substance.

It is to be understood that other additions to the synthetic or organic materials may be used. For example, whilst the above discussion looked at the use of organic abrasive particles or coating particles with such oils and fragrances, one may also add colouring to the abrasive particles whether they are synthetic or organic. This can result in the skin not only being abraded but also leaving a residue of oils/fragrance/colours to the specification of the consumer. The colours may also be natural colours form the organic material or may be colouring added to the particles. Thus it is to be understood that the additive to the abrasive particle are not intended to be limited to those described above. They may be various, such as tea tree oils and essences, colourings and so on. The only requirement would be that as a collection of particles they cannot posses a coating that will provide for adhesion between the different particles.

One can therefore see that the present invention teaches the use of organic or organic coated particles together with an apparatus that can accommodate one or more different supply containers that may require one or more recovery containers as well as one or more pneumatic sources. This provides for a hitherto unavailable choice in the treatment of skin both for cosmetic and medical purposes.

For example, one may first wish to be treated with a harsh particle to remove quickly and efficiently top surface skin layers. Subsequently one may wish to use the gentler but still slightly abrasive organic particles that compensate for any serious abrasion whilst imparting to the skin substances that help protect and nourish it.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus.

1. An apparatus for providing treatment of a surface such as skin including:
a pneumatic source operatively connected to a recovery container and to a hand tool;
a plurality of supply containers including particulates;
a valve selectively connecting said pneumatic source, recovery container and hand tool to a selected one of said supply containers;
whereby in operation said pneumatic source provides for an air flow through the recovery container, hand tool and the selected supply container in communication with same, drawing said particles from the supply container through the hand tool and into the recovery container, said hand tool positioned on a surface to be treated and including an aperture so located that particles passing through said hand tool are caused to impinge on the surface thereby treating it.

2. An apparatus as in claim 1 further including a plurality of recovery containers whereby each supply container has a corresponding unique recovery container that it is in communication with.

3. An apparatus as in claim 1 or claim 2 further including a plurality of hand tools each hand tool being associated with a unique supply container.

4. An apparatus as in any one of the above claims wherein each supply container contains different particles.

5. An apparatus as in claim 5 wherein at least one supply container includes aluminium oxide particles and at least one other supply container includes aluminium oxide particles whose size is on the average greater than those in the other.

6. An apparatus as in claim 5 wherein at least one said supply container includes organic type particles.

7. An apparatus as in claim 6 wherein said organic type particles are made from ground up nut shells chosen from the selection including but not limited to walnut, hazelnut, brazil and almond nut shells or a mixture thereof.

8. An apparatus as in claim 6 wherein said organic type particles are made from ground up stone fruit kernels chosen from the selection but not limited to peach, apricot, nectarine, peacherine, plum, cherry stones or a mixture thereof.

9. An apparatus as in any one of the above claims wherein the pneumatic source is a vacuum pump.

10. An apparatus as in claim 9 wherein the vacuum pump includes a regulator so as to control the air pressure within the apparatus.

11. An apparatus as in any one of the above claims further including a second pneumatic source selectively and operatively connected to another supply container.

12. An apparatus as in claim 11 wherein said second pneumatic source is a compressor.

13. An apparatus as in claim 12 wherein said compressor can variably control the air pressure fed into the supply container.

14. An apparatus as in any one of the above claims wherein there are at least three supply containers each including different particles to assist in treatment of the skin.

15. An apparatus as in any one of the above claims wherein at least some of said particles are coated with fragrant materials.

16. An apparatus as in any one of the above claims wherein at least some of said particles are coated with a colour.

17. An apparatus for providing treatment of a surface including:
a pneumatic source operatively connected to a recovery container, to a hand tool, and to a supply container said supply container housing organic particulates;
whereby in operation said pneumatic source provides for an air flow through the recovery container, hand tool and the supply container in communication with same, drawing said organic particles from the supply container through the hand tool and into the recovery container, the hand tool being positioned on a surface to be treated and including an aperture so located that particles passing through said hand tool are caused to impinge on the surface thereby treating it.

18. An apparatus as in claim 17 wherein said organic particles include a surface film pat of which remains in the surface being treated.

19. A method of treatment of human skin including directing an airflow including organic particles onto the skin to be treated.

20. A method of treating human skin including:
using an air flow to carry particles through a hand tool, said hand tool including an aperture which is so located that upon placement on the skin the particles are caused to impinge on it and wherein said particles are organic type particles.
